(19) United States
(12) Patent Application Publication
(10) Pub. No.: US 2013/0165545 A1
(43) Pub. Date: Jun. 27, 2013

(54) BONDING TOGETHER RESIN NANO CERAMIC BLOCKS

(71) Applicant: Vincent J. Morgan, Boston, MA (US)

(72) Inventor: Vincent J. Morgan, Boston, MA (US)

(21) Appl. No.: 13/336,762

(22) Filed: Dec. 23, 2011

(51) Int. Cl. A61K 6/08; B82Y 30/00

(52) U.S. Cl. 523/115; 977/773

(57) ABSTRACT

First and second smaller blocks of resin nano ceramic material are bonded together to form a larger resin nano ceramic block.

The present invention relates in general to bonding together blocks of resin nano ceramic material and more particularly concerns bonding together smaller blocks to form a larger block, especially useful for dental prostheses.

### BACKGROUND OF THE INVENTION

Fabrication of larger homogeneous resin nano ceramic blocks have been difficult to achieve because of the inclusion of cracks, fissures or voids during the fabrication process of polymerization.

### SUMMARY OF THE INVENTION

It has been discovered that adhering smaller blocks together avoids cracks, fissures and voids that occur when fabricating larger or multicolored layered blocks. Each smaller block is less than a maximum span from one edge to an opposite edge, typically being the major diameter of an elliptical or circular block and free of cracks, fissures or voids so that the maximum span of the assembly of the smaller blocks adhered or bonded together is free of cracks, fissures, or voids.

Various light-cured methacrylate-based restoratives are suitable as adhesive materials for bonding smaller blocks together for the fabrication of larger blocks, colored layered blocks and various geometrically shaped blocks. The invention achieves exceptional bond strengths to facilitate the fabrication of larger and varied geometrical shaped blocks, disks or pucks which could then be milled and overcomes the current difficulties in processing larger or varied shaped resin nano ceramic materials for CAD/CAM processing.

The larger pieces allow for multiple crowns or units to be milled at one time. U-shaped blocks allow for considerable savings of material formulating a prosthesis in the form of a dental arch.

It is an important object of the invention to avoid cracks, fissures and voids while fabricating larger or multi-colored layered resin nano ceramic blocks.

Other features, objects and advantages of the invention will become apparent from the following detailed description when read in connection with the accompanying drawing in which:

### BRIEF DESCRIPTION OF THE DRAWING

FIG. 1 is a top view of an embodiment of the invention formed with three small blocks bonded together;

FIG. 2 is a bottom view of the embodiment of FIG. 1;

FIG. 3 is a top view of another embodiment formed by three small blocks bonded together; and

FIG. 4 and FIG. 5 are perspective views of a three-tooth prosthetic bridge.

### DETAILED DESCRIPTION

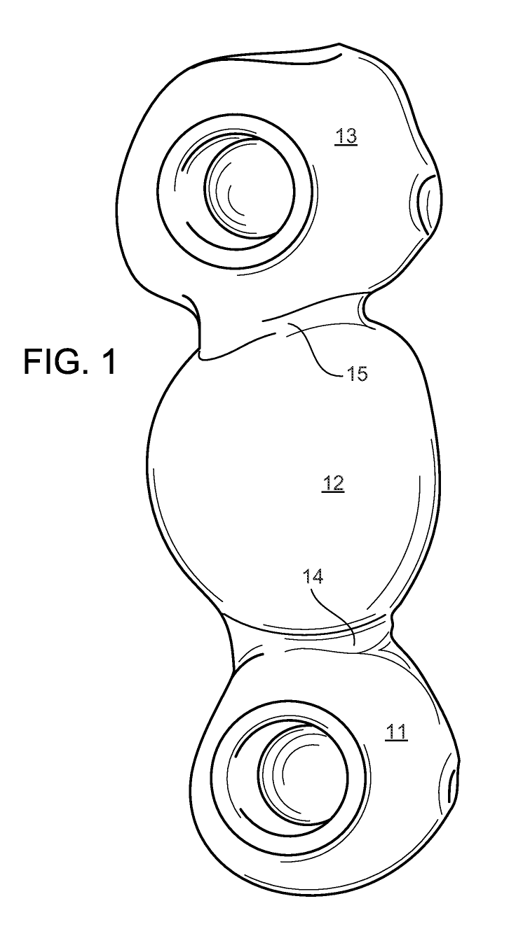

With reference now to the drawing and more particularly FIG. 1 thereof, there is shown an embodiment of the invention formed by three small discs 11, 12 and 13 bonded together at 14 and 15 with a light-cured methacrylate-based restorative.

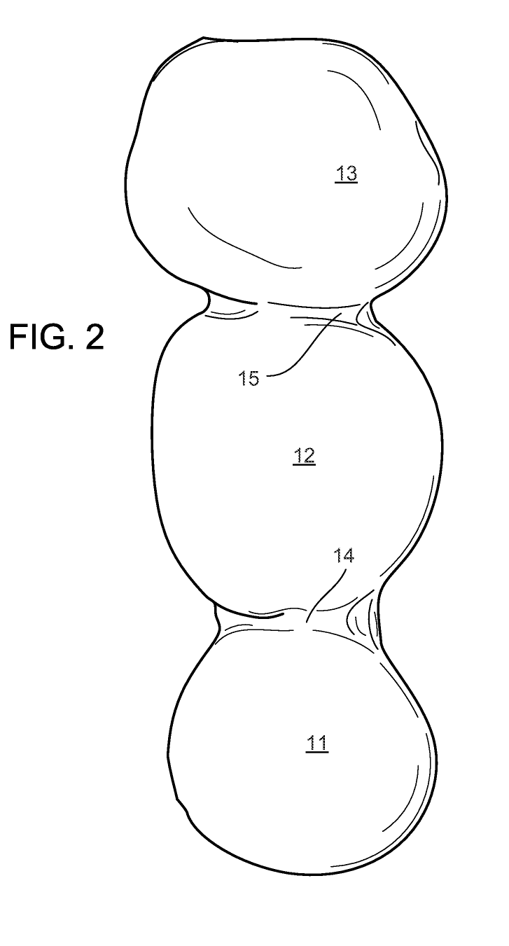

Referring to FIG. 2, there is shown a bottom view of the embodiment of FIG. 1.

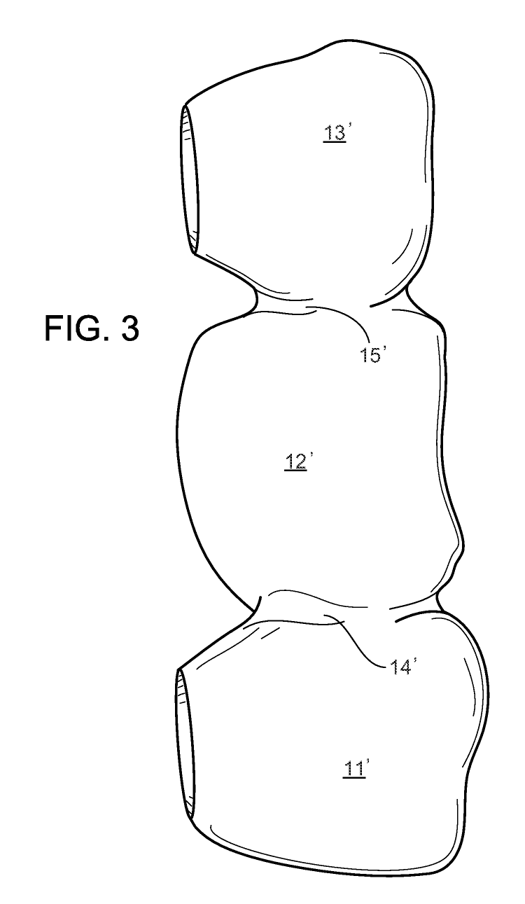

Referring to FIG. 3, there is shown a plan view of another embodiment of the invention comprising discs 11′ 12′ and 13′ bonded together at 14′ and 15′.

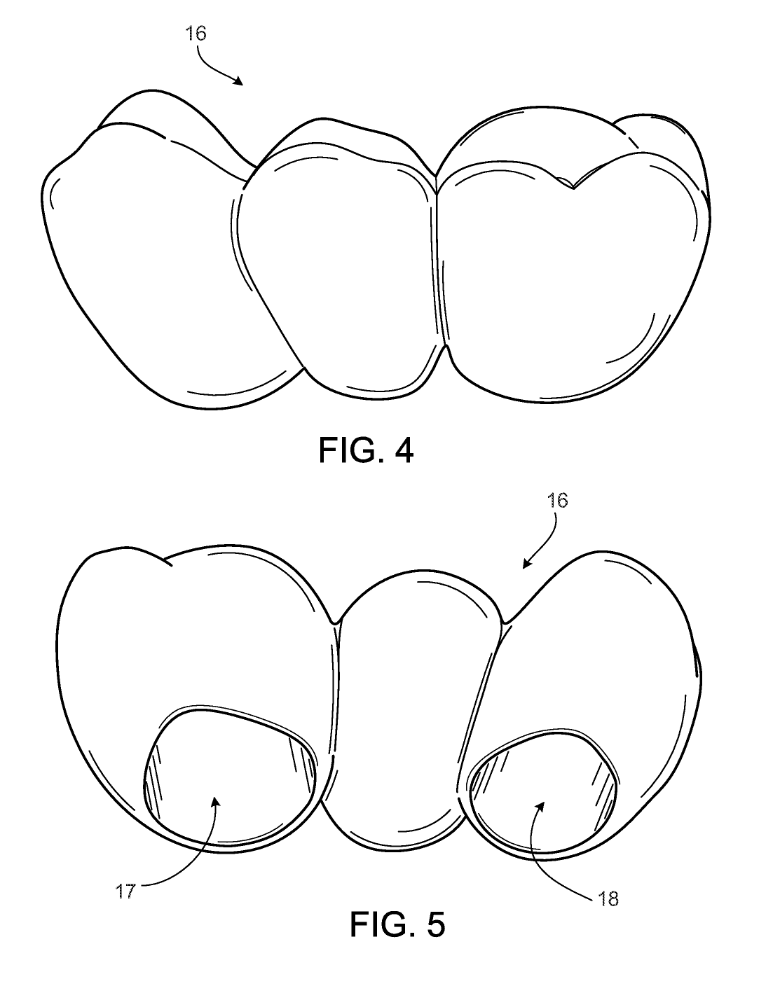

Referring to FIG. 4 there is shown a perspective view of a three-tooth prosthetic bridge 16 formed by first bonding at least two blocks together and then milling the bonded blocks to form prosthetic teeth 16A, 16B and 16C. Referring to FIG. 5 there is shown a perspective view of the cervical side of prosthetic bridge 16 formed with bores 17 and 18 for accepting respective abutments in a patient and a pontics to fill the edentulous space between to secure prosthetic bridge 16 in the mouth of the patient in a location formerly occupied by all natural teeth. The abutment may be natural teeth shaped to fill the bores or implant abutments. The edentulous space is the space formerly occupied by a natural tooth.

An example of steps for bonding resin nano ceramic CAD/CAM blocks with methacrylated-based light-cured restoratives follows:

1. Roughen the resin nano ceramic site to be added to with a coarse diamond or stone, or air abrade (sandblast with 50 μm aluminum oxide particles at 80 psi at an approximately 0.39 inches distance). Clean the roughened block in an ultra-sonic cleaner with normal detergent type cleaner.

2. Brush on a one-step self-etch adhesive system, which could be either a 1 or 2-component system. If 1-component, most common chemical composition is hydroxyethyl methacrylate (HEMA), initiator and inhibitor, with 1 to 10% weight fillers. 2-components commonly have approximately 50% water and a co-solvent, Mono-methacrylates, and Dimethacrylates. After being brushed, blow the area gently dry for 2 to 5 seconds for solvent evaporation.

3. Self-cured systems should be left indisturbed for 1 minute until step 4. When light is needed for light or dual-cured, curing time is usually 10 seconds.

4. Apply any light-cured methacrylate-based restorative material to bond the blocks and then light-cure according to the instructions for use.

5. Finish and polish with polishing discs of increasingly finer grits followed by a bristle brush and white diamond or diamond paste. Buff to a high shine with a small muslin rag wheel.

The roughening step may be with a coarse diamond or stone, or air abrade (50Π aluminum oxide sandblast) and then clean in an ultra-sonic cleaner with a normal detergent. The brush on step may be with a 3M ESPE ADPER Easy Bond Self-Edge adhesive to the roughened area and gently blown dry for 2 to 5 seconds. The adhesive may be light-cured typically for 10 seconds. The finishing and polishing may be shaped and smoothed with 3M ESP SOF-LEX contouring and polishing discs and then polishing with a bristle brush and polishing compound.

The CAD/CAM blocks may by commercially available 3M Lava Ultimate whose structure is a resin nano ceramic; specifically composed by approximately 79% (by weight) nano ceramic particles bound in the resin matrix, the ceramic particles being made up of three different ceramic fillers that reinforce a highly crossed linked polymeric matrix. The fillers are a combination of nonagglomerated/non-aggregated 20 nanometer silica filler, nonagglomerated/non-aggregated for this 4 to 11 nanometer zirconia and aggregated zirconia/silica cluster filler (comprised of 20 nanometer silica and 4 to 11 nanometer zirconia particles.

It is evident that those skilled in the art may now make numerous uses and modifications of the specific structures and techniques disclosed herein without departing from the inventive concepts. Consequently, the invention is to be construed as embracing each and every novel feature and novel combination of features present in or possessed by the structures and techniques herein disclosed and limited solely by the spirit and scope of the appended claims.

What is claimed is:

1. At least first and second smaller blocks bonded together to form a larger block, each smaller block being a resin nano ceramic material subject to cracks, fissures or voids during the fabrication process of polymerization when the maximum span of a block is greater than that of each smaller block to form a larger block free of cracks, fissures or voids with a maximum span greater than that of each smaller block.

2. The structure of claim 1 comprising first, second and third of said smaller blocks bounded together with the second smaller block between the first and third smaller blocks.

3. The structure of claim 1 milled to form at least one prosthetic tooth.

4. The structure of claim 1 milled to form a prosthetic multi-tooth bridge having a cervical side and pontics to fill the edentulous space bridged.

5. The structure of claim 4 formed with at least one bore in the cervical side for accommodating an abutment.

6. The structure of claim 2 milled to form at least one prosthetic tooth.

7. A method of making a larger resin nano ceramic CAD/CAM block having a maximum span greater than that of at least two smaller blocks free of cracks, fissures, or voids comprising bonding at least first and second of the smaller nano ceramic blocks together.

8. The method of claim 7 and further including bonding the first and second smaller blocks together with an adhesive.

9. The method of claim 7 and further including milling the larger block to form at least one prosthetic tooth.

10. The method of claim 7 and further including milling the larger block to form a prosthetic multi-tooth bridge.
